(19) United States
(12) Patent Application Publication
(10) Pub. No.: US 2025/0383339 A1
(43) Pub. Date: Dec. 18, 2025

(54) DESIGN AND APPLICATION OF NOVEL LIGAND-INDUCED SPLIT-PROTEIN SYSTEMS

(71) Applicant: ARIZONA BOARD OF REGENTS ON BEHALF OF THE UNIVERSITY OF ARIZONA, Tucson, AZ (US)

(72) Inventors: Indraneel Ghosh, Tucson, AZ (US); Samuel Sugerman, Tucson, AZ (US); Brittany M. Stankavich, Tucson, AZ (US)

(21) Appl. No.: 18/878,486

(22) PCT Filed: Jun. 27, 2023
(86) PCT No.: PCT/US2023/069176

§ 371 Date: Dec. 23, 2024

Related U.S. Application Data
(60) Provisional application No. 63/355,962, filed on Jun. 27, 2022.

(51) Int. Cl. G01N 33/50; G01N 33/68

(52) U.S. Cl. CPC G01N 33/5023; G01N 33/6845

(57) ABSTRACT

A biotechnological chemically induced dimerization (CID) tool with potential technological and therapeutic applications thereof. These CID systems are used in biological research to control numerous outputs in living organisms, such as inducing the activation of a specific protein, protein localization, and inducing transcription. A novel CID system is based on the recognition that ligand binding may stabilize a newly designed ternary complex.

### CROSS-REFERENCES TO RELATED APPLICATIONS

This application claims benefit of U.S. Provisional Application No. 63/355,962 filed Jun. 27, 2022, the specification of which is incorporated herein in its entirety by reference.

### STATEMENT REGARDING FEDERALLY SPONSORED RESEARCH OR DEVELOPMENT

This invention was made with government support under Grant No. GM115595 awarded by National Institutes of Health. The government has certain rights in the invention.

### REFERENCE TO A SEQUENCE LISTING

The contents of the electronic sequence listing (ARIZ_22_15_PCT_Sequence_Listing.xml; Size: 38,896 bytes; and Date of Creation: Jun. 27, 2023) is herein incorporated by reference in its entirety.

### FIELD OF THE INVENTION

The present invention features systems, compositions, and methods that allow for the oligomerization and/or modulation of target proteins.

### BACKGROUND OF THE INVENTION

Chemically Induced Dimerization (CID) is a biotechnological method where two or more proteins can bind each other and form a ternary or higher complex only in the presence of a specific small molecule or another dimerizing ligand. These CID systems are used in biological research to control numerous outputs in living organisms, such as inducing the activation of a specific protein, protein localization, and inducing transcription. These different ligands in CID systems can be engineered to induce activation of protein outcomes that can be used to control a variety of signaling pathways and their related physiological outputs and thus be used to study biology and treat diseases. The present invention features a novel approach for the engineering of new CID systems based on the recognition that ligand binding may stabilize a newly designed ternary complex.

### SUMMARY OF THE INVENTION

It is an objective of the present invention to provide systems, compositions, and methods that allow for the oligomerization and/or modulation of target proteins, as specified in the independent claims. Embodiments of the invention are given in the dependent claims. Embodiments of the present invention can be freely combined with each other if they are not mutually exclusive.

In some embodiments, the present invention features a split protein system. For the split protein system, a first protein is split into a first protein fragment and a second protein fragment, and a target protein is split into a first target fragment and a second target fragment. The protein system comprises a first split protein complex comprising the first target fragment operatively linked to the first protein fragment, a second split protein complex comprising the second target fragment operatively linked to the second protein fragment, and a ligand capable of chemically inducing dimerization of the first split protein complex and the second split protein complex by operatively linking the first protein fragment and the second protein fragment. In some embodiments, the ligand can interact with the first protein fragment and the second protein fragment to modulate the activity of the target protein.

In other embodiments, the present invention features a method of chemically induced dimerization of a target protein. The method may comprise splitting a first protein into a first protein fragment and a second protein fragment, splitting the target protein into a first target fragment and a second target fragment, and operatively linking the first target fragment to the first protein fragment to form a first split protein complex, operatively linking the second target fragment to the second protein fragment to form a second split protein complex, and contacting the first split protein complex and the second split protein complex with a ligand. In some embodiments, the ligand chemically induces dimerization of the first split protein complex and the second split protein complex by operatively linking the first protein fragment and the second protein fragment.

In some embodiments, the present invention features a method for multimerizing a target protein in cells. The method may comprise providing cells that contain a) a first recombinant nucleic acid encoding a first chimeric protein, wherein the first chimeric protein comprises a first protein fragment operatively linked to a first target fragment, and b) a second recombinant nucleic acid encoding a second chimeric protein, wherein the second chimeric protein comprises a second protein fragment operatively linked to a second target fragment. The method may further comprise contacting the aforementioned cells with a ligand. The ligand chemically induces multimerization of the first chimeric protein and the second chimeric protein by operatively linking the first protein fragment and the second protein fragment.

One of the unique and inventive technical features of the present invention is the use of a ligand-gated split-protein system. Without wishing to limit the invention to any theory or mechanism, it is believed that the technical feature of the present invention advantageously allows for the modulation of a target protein within a cell. None of the presently known prior references or work has the unique, inventive technical feature of the present invention.

Furthermore, the prior references teach away from the present invention. For example, prior references teach of only two discrete classes of chemically-induced dimerization. The first class uses a ternary complex that has been observed in nature and consists of two separate domains and a chemical ligand. The second class uses an observed protein ligand interaction in conjunction with another protein domain designed to interact with the protein ligand complex but not with either component (i.e., the protein or ligand component) alone.

Contrastingly, the present invention has created a novel class (e.g., a third class) of chemically-induced dimerization. This novel class of CID described herein uses an observed protein ligand interaction. The protein is split (e.g., fragmented at a split site) into at least two stand-alone fragments, which then oligomerize with the addition of the ligand.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows partial alignment of Bcl-2 family proteins. This shows gaps in the alignment that identified insertion sites.

FIG. 1B shows a topological representation of Bcl-xL. Sites chosen for fragmentation are depicted as dashed lines and are labeled as “1” for the α3-α4 split, “2” for the α4-α5 split, and “3” for the α5-α6 split. Fragmentation sites are additionally represented with scissors.

FIG. 1C shows a three-dimensional representation of Bcl-xL bound to the BH3 BAD (PDB ID 2BZW) with fragmentation sites depicted with the same numbers as FIG. 1B.

FIG. 2A shows a schematic of the split-reporter setup used in the presently claimed invention.

FIG. 2B shows plasmids used to generate the three separate split sites outlined in FIGS. 1A-1C. See SEQ ID NO: 13-24.

FIG. 2C shows the split luciferase assay results at the Bcl-xL split sites indicated on the x-axis. Cells were transiently transfected with 50 ng of each NFluc and CFluc, and treated with either DMSO (white) or five μM ABT-263 (gray). Fold change is indicated as a measure of small-molecule dependence of the luminescence.

FIG. 3A shows the split luciferase assay results using the α3-α4 site and treating with 50 nM ABT-263. Bcl-xL R139A (gray) and Bcl-xL WT (black) respond similarly to ABT-263 at five ng transfections of each FLuc construct.

FIG. 3B shows results from the split luciferase assay using the same transfections of Bcl-xL WT (black) and Bcl-XL R139A (gray) as in 7A but treating instead with 50 nM A-1155463. Data are presented as the average of quadruplicates, with the replicates divided after transfection.

FIG. 4A shows a schematic of split Src kinase dimerized by split Bcl-XL in the presence of Bcl-xL ligands such as ABT-263. See SEQ ID NO: 25-28.

FIG. 4B shows an anti-phosphotyrosine western blot of split-Src controlled by split Bcl-xL, with constructs treated with either DMSO or Bcl-xL inhibitors ABT-263, A-1155463, or ABT-737.

FIG. 5 shows an anti-phosphotyrosine western blot of split-Src controlled by the ABI/PYL CID system, with constructs treated with either DMSO, abscisic acid (ABA) or abscisic acid plus ABT-263. The split kinase alone exhibits low phosphorylation (lane 1), and the addition of ABA turns the kinase activity on (lane 2). Addition of ABT-263 with ABA to the fragments causes a lesser amount of kinase turn-on (lane 4), suggesting that ABT-263 may be inhibiting Src kinase activity in some fashion.

FIG. 6 shows a graph of the determination of the timing of L-GLO incubation. 50 ng of each FLuc half was treated with either ABT-737 (circles) or DMSO (triangles). RLU was measured for 100 minutes following the addition of L-GLO. The top-right shows the signal/background ratio at each time point. Based on these data, the assay was measured after the most rapid changes in signal/noise ratio, and at least 25 minutes elapsed after adding L-GLO before luminescence values were recorded. All data presented herein follow that timing.

FIG. 7A shows a graphical protocol of titration experiments to determine the optimal response to treatment conditions.

FIG. 7B shows a graph of signal per microgram of duplicate treatment biological replicates measured in quadruplicate. This shows low effects on signal regardless of FBS percentage in the treatment period.

FIG. 7C shows light microscopy photos of HEK293T cells in various treatment conditions.

FIG. 8A shows a partial alignment of DHFR variants from different species and identifies several non-conserved regions in eDHFR.

FIG. 8B shows a topological representation with all identified non-conserved eDHFR sites depicted as dashed lines and are labeled as “1” for the α1-β3 split, “2” for the α4-β7 split, and “3” for the β9-β10 split. Fragmentation sites are additionally represented with scissors.

FIG. 8C shows non-conserved sites from FIG. 8B identified on the recent crystal structure of eDHFR bound to trimethoprim (PDB 6XG5).

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Referring to the figures, in some embodiments, the present invention features a split protein system. For the split protein system, a first protein is split into a first protein fragment and a second protein fragment, and a target protein is split into a first target fragment and a second target fragment. The protein system comprises a first split protein complex comprising the first target fragment operatively linked to the first protein fragment, a second split protein complex comprising the second target fragment operatively linked to the second protein fragment, and a ligand capable of chemically inducing dimerization of the first split protein complex and the second split protein complex by operatively linking the first protein fragment and the second protein fragment. In some embodiments, the ligand can interact with the first protein fragment and the second protein fragment to modulate the activity of the target protein.

As used herein, the “activity” of a target protein may be used interchangeably with the “function” of a target protein and may refer to the ability of a protein (e.g., an enzyme) to catalyze a reaction. Thus, to modulate the activity of a target protein may refer to an increase or decrease in a protein's (e.g., enzyme's) ability to catalyze a reaction. For example, methods described herein may increase or decrease the kinase activity (i.e., the ability to phosphorylation a substrate) of a target protein (e.g., a kinase protein, e.g., Src).

The methods described herein may also allow for ligand-induced localization of a target protein. In some embodiments, a ligand may induce localization of a target protein by tethering said protein somewhere in the cell upon the addition of said ligand. Without wishing to limit the present invention, it is believed that induced localization by the ligand allows for additional regulation of the target protein (e.g., extracellular or nuclear localization, membrane binding, or trafficking).

In some embodiments, the first protein comprises a plurality of split sites. The first protein may be split at at least one of the split sites. In some embodiments, the first protein may be split at one split site (e.g., into a first protein fragment and a second protein fragment). In other embodiments, the first protein may be split at two split sites (e.g., into a first protein fragment, a second protein fragment, and a third protein fragment). In further embodiments, the first protein may be split at three split sites (e.g., into a first protein fragment, a second protein fragment, a third protein fragment, and a fourth protein fragment). Without wishing to limit the present invention to any theory or mechanism, it is believed that splitting the first protein into more than two fragments (i.e., a first fragment and a second fragment) may reduce background dimerization (e.g., dimerization of the first protein in the absence of the ligand).

In embodiments where the first protein is split at two or more split sites (e.g., split into three or more fragments), the fragments may either spontaneously oligomerize or oligomerize in the presence of a ligand. In the aforementioned embodiment, a linker may not be required to link a fragment of the first protein to a fragment of the target protein. In a separate embodiment, a first protein may be split into three or more fragments, and non-contiguous fragments may be operatively linked together via a linker such that the remaining fragment(s) may oligomerize in the presence of a ligand or binding partner.

In some embodiments, the target protein comprises a plurality of split sites. The target protein may be split at at least one of the split sites. In some embodiments, the target protein may be split at one split site (e.g., into a first target fragment and a second target fragment). In other embodiments, the target protein may be split at two split sites (e.g., into a first target fragment, a second target fragment, and a third target fragment). In further embodiments, the target protein may be split at three split sites (e.g., into a first target fragment, a second target fragment, a third target fragment, and a fourth target fragment). In accordance with the present invention, the target protein does not need to be split into the same number of fragments as the first protein. For example, the first protein may be split into three fragments (e.g., into a first protein fragment, a second protein fragment, and a third protein fragment), whereas the target protein may only be split into two fragments (e.g., into a first target fragment and a second target fragment).

As used herein, a “split site” may refer to a stretch of non-homologous or different amino acids identified from the primary sequence alignment of closely related species encoding a similar gene. This stretch of amino acids can also be mapped to a solvent accessible loop region within the crystal or NMR structure of the protein of interest. In some embodiments, the split site is determined by sequence dissimilarity or differences in sequence length. In some embodiments, split sites may be clustered in surface-exposed loops with disordered secondary structures. As used herein, “fragment site,” “split site,” or “fragmentation site” may be used interchangeably.

As used herein, “sequence dissimilarity” may refer to a region within a protein's primary sequence that is non-homologous or different when aligned to a sequence of a closely related species that expresses a similar gene.

In some embodiments, the ligand is a small molecule ligand. In some embodiments, the ligand is an inhibitor of the first protein. In some embodiments, the ligand comprises a peptide or a full-length protein. In some embodiments, the ligand comprises a metabolite or an intermediate metabolite. In some embodiments, the ligand comprises a natural product.

In some embodiments, the first target fragment is operatively linked to the first protein fragment via a linker. In other embodiments, the second target fragment is operatively linked to the second protein fragment via a linker. In some embodiments, the linkers comprise flexible linkers. The lengths of the linkers can range from about 10 residues to about 30 residues or more. In some embodiments, the linker comprises about 5 to 35 residues, or about 5 to 25 residues, or about 5 to 15 residues, or about 10 to 35 residues, or about 10 to 25 residues, or about 10 to 15 residues, or about 15 to 35 residues, or about 15 to 25 residues, or about 25 to 35 residues. Any linker known in the art that creates enough space between the linked fragments (e.g., the first protein fragment and the first target fragment) to prevent steric occlusion may be used in accordance with the present invention.

In some embodiments, the first protein fragment comprises an N-terminus of the first protein, and the first target fragment comprises an N-terminus of the target protein. The first protein fragment may be linked to the N-terminus of the first target fragment. In some embodiments, the second protein fragment comprises a C-terminus of the first protein, and the second target fragment comprises a C-terminus of the target protein. The second protein fragment may be linked to the C-terminus of the second target fragment.

In a non-limiting embodiment, the first protein may comprise a Bcl-xL protein. In some embodiments, the Bcl-xL protein is mutant Bcl-xL R139A. Other mutations that allow for selective binding of the ligand to the first protein may be used in accordance with the systems of the present invention. In some embodiments, the first protein may comprise any protein with at least one viable (or potential) split site and at least one known ligand (e.g., a small molecule ligand).

In some embodiments, the first protein fragment is N-Bcl-XL (aa 1-112), and the second protein fragment is C-Bcl-xL (aa 113-209). In some embodiments, the first protein fragment is N-Bcl-xL (aa 1-135), and the second protein fragment is C-Bcl-xL (aa 136-209). In some embodiments, the first protein fragment is N-Bcl-xL (aa 1-164), and the second protein fragment is C-Bcl-XL (aa 165-209).

In a non-limiting embodiment, the ligand is a Bcl-xL ligand (e.g., A-1155463, ABT-263, or ABT-737). Non-limiting examples of Bcl-XL ligands include but are not limited to A-1331852, A-1155463, ABT-263, ABT-737, WEHI-539, WEHI-539 hydrochloride, Gambogic Acid, TW-37, ABT-737, HA14-1, BH3 peptides, or a combination thereof. In a non-limiting embodiment, the ligand is a Bcl-xL inhibitor.

In some embodiments, the target protein is any functional protein with at least one viable (or potential) split site, as described herein. Non-limiting examples of target proteins may include but are not limited to kinases, proteases, luciferases, fluorescent proteins, or CRISPR-Cas assemblies. In a non-limiting embodiment, the target protein is a tyrosine kinase (e.g., Src).

According to other embodiments, the present invention features a split protein system. The protein system comprises a first protein split into a first protein fragment and a second protein fragment and a ligand capable of chemically inducing dimerization of the first protein by operatively linking the first protein fragment and the second protein fragment. In some embodiments, the ligand can interact with the first protein fragment and the second protein fragment to modulate the activity of the first protein. This split protein system can be used for chemically induced dimerization of other split proteins.

According to some other embodiments, the present invention features a method of chemically induced dimerization of a target protein. The method may comprise splitting a first protein into a first protein fragment and a second protein fragment, splitting the target protein into a first target fragment and a second target fragment, operatively linking the first target fragment to the first protein fragment to form a first split protein complex, operatively linking the second target fragment to the second protein fragment to form a second split protein complex, and contacting the first split protein complex and the second split protein complex with a ligand. In some embodiments, the ligand chemically induces dimerization of the first split protein complex and the second split protein complex by operatively linking the first protein fragment and the second protein fragment.

In some embodiments, the present invention features a method of chemically induced dimerization of a target protein. The method may comprise forming a first split protein complex by operatively linking a first protein fragment from a first protein to a first target fragment from a target protein and forming a second split protein complex by operatively linking a second protein fragment from the first protein to a second target fragment from the target protein. The method may further comprise contacting the first split protein complex and the second split protein complex with a ligand. The ligand chemically induces dimerization of the first split protein complex and the second split protein complex by operatively linking the first protein fragment and the second protein fragment.

The present invention may also feature a split protein system comprising a first recombinant nucleic acid encoding a first chimeric protein, wherein the first chimeric protein comprises a first protein fragment operatively linked to a first target fragment and a second recombinant nucleic acid encoding a second chimeric protein, wherein the second chimeric protein comprises a second protein fragment operatively linked to a second target fragment. The system may further comprise a ligand capable of chemically inducing multimerization (e.g., dimerization) of the first chimeric protein and the second chimeric protein by operatively linking the first protein fragment and the second protein fragment.

The present invention may also feature a method for multimerizing a target protein in cells. The method may comprise providing cells that contain a first recombinant nucleic acid encoding a first chimeric protein and a second recombinant nucleic acid encoding a second chimeric protein. The first chimeric protein may comprise a first protein fragment operatively linked to a first target fragment, and the second chimeric protein may comprise a second protein fragment operatively linked to a second target fragment. The method may further comprise contacting the cells with a ligand that chemically induces multimerization (e.g., dimerization) of the first chimeric protein and the second chimeric protein by operatively linking the first protein fragment and the second protein fragment.

In some embodiments, the method comprises incubating the aforementioned cells with a ligand for a period of time. For example, the aforementioned cells may be incubated with the ligand for about five minutes, or about ten minutes, or about twenty minutes, or about twenty-five minutes, or about thirty minutes, or about forty minutes. In other embodiments, the ligand may be added to the aforementioned cells immediately before lysis of said cells.

In some embodiments, the cells described herein are mammalian cells. In some embodiments, the cells described herein are present in a whole organism (e.g., a mammal), and contacting the cells with the ligand comprises administering the ligand to the organism (e.g., the mammal).

### Example

The following is a non-limiting example of the present invention. It is to be understood that said example is not intended to limit the present invention in any way. Equivalents or substitutes are within the scope of the present invention.

In some embodiments, the present invention features the creation of the first split-Bcl-xL proteins that are stabilized by small molecule ligands. The present invention further demonstrates that specific split-Bcl-xL based CID systems can be utilized for controlling the function of different enzymes by adding an exogenous small molecule to live mammalian cells. This approach for CID generation may likely be generalizable and may be extended to many different protein: ligand complexes and represents a potentially significant advance in protein engineering technology as CIDs are used for many different applications.

This new approach for designing ligand-induced split protein dimerization was tested in the context of the Bcl-xL family proteins that have been shown to bind peptides and designed small molecule inhibitors. Based on the interaction with both BH3 peptides and small-molecule ligands, an appropriately designed fragmented Bcl-XL protein, N-Bcl-xL and C-Bcl-XL, could be re-assembled or dimerized in the presence of a high-affinity binder. Ternary complexes were designed to include N-Bcl-xL, C-Bcl-xL, and either a BH3 peptide or a small-molecule Bcl-XL inhibitor as a ligand. The hypothesis was that the ternary complex would be more thermodynamically stable than any of the monomers or possible dimers.

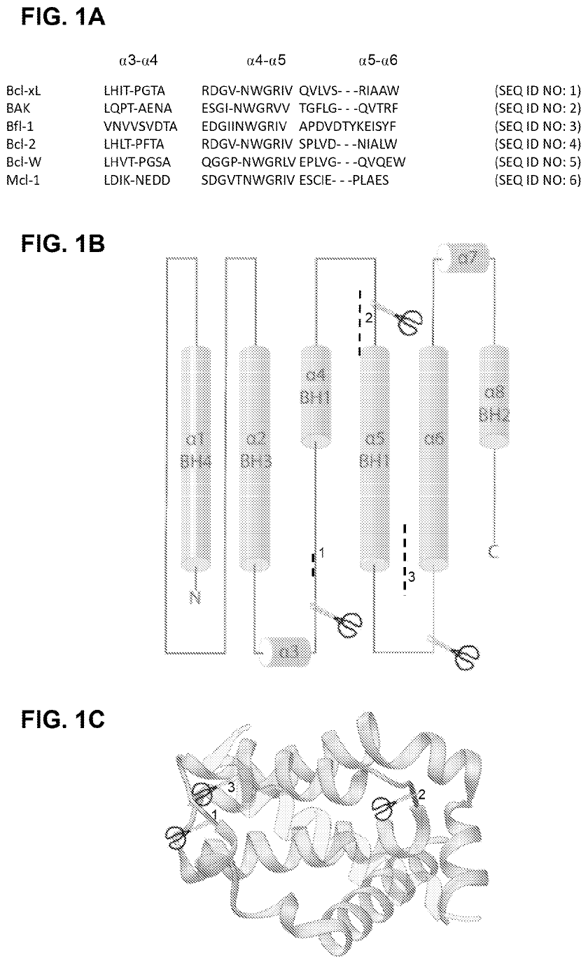

Bcl-xL was aligned with the other Bcl-2 family proteins for which affinity data has been previously generated. Alignment of the Bcl-2 family of proteins suggested that there were multiple loops in the protein that were non-homologous (FIG. 1A). Previous research has shown that non-homologous loops may be amenable to fragmentation. Mapping these sites to both topological representations and three-dimensional crystal structures of Bcl-xL 209 showed that two of the three split sites identified were in flexible loops on the crystal structure (FIGS. 1B and 1C). The third potential split site, between α5 and α6, was on the first turn of an alpha helix in the crystal structures (FIG. 1C, “3”). For the ligand-gated split-protein design, the single mutant Bcl-XL R139A, a less promiscuous peptide binder, may be implemented.

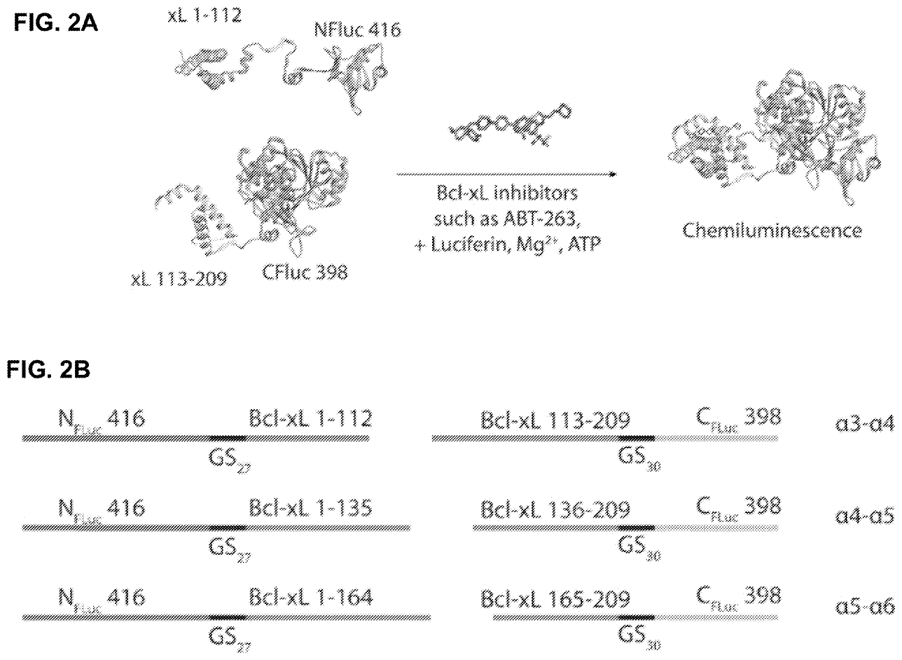

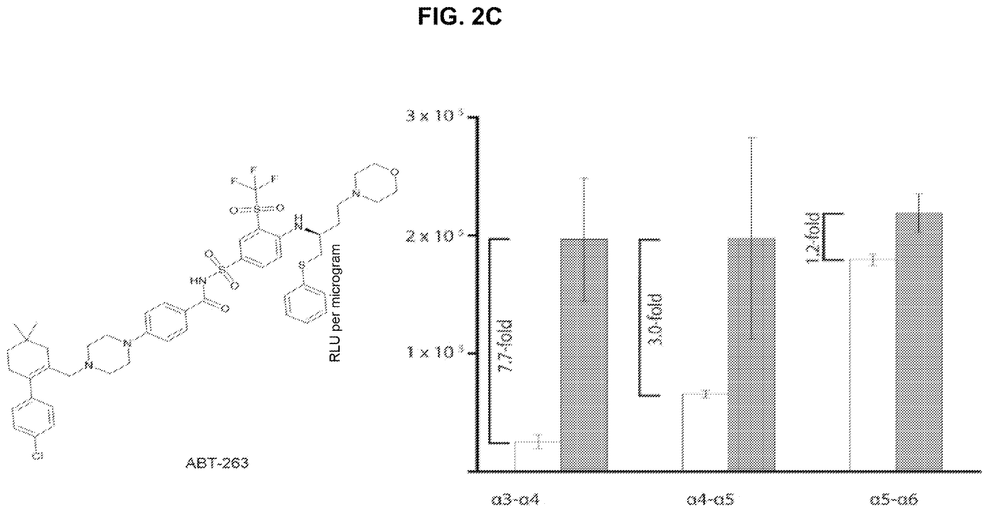

To design split Bcl-xL, a suitable readout was required to measure successful ligand-dependent reassembly. A split-luciferase complementation assay was used, which was originally identified by rapamycin-induced complementation of FKBP and FRB. The split protein constructs were designed such that split-firefly luciferase-dependent luminescence would only be generated upon dimerization of N-Bcl-xL and C-Bcl-xL fragments either in the absence or presence of ligands (FIG. 2A). The split-protein constructs were created for each of the Bcl-xL split sites (FIG. 2B) and tested by transient transfection into HEK293T cells with the addition of either a Bcl-xL ligand or DMSO. The results of the assay demonstrate Bcl-xL 209 reassembly in each of the three split sites tested and, more importantly, ligand-dependent signal for the split sites corresponding to the loops between α3-α4 and α4-α5 (FIG. 2C).

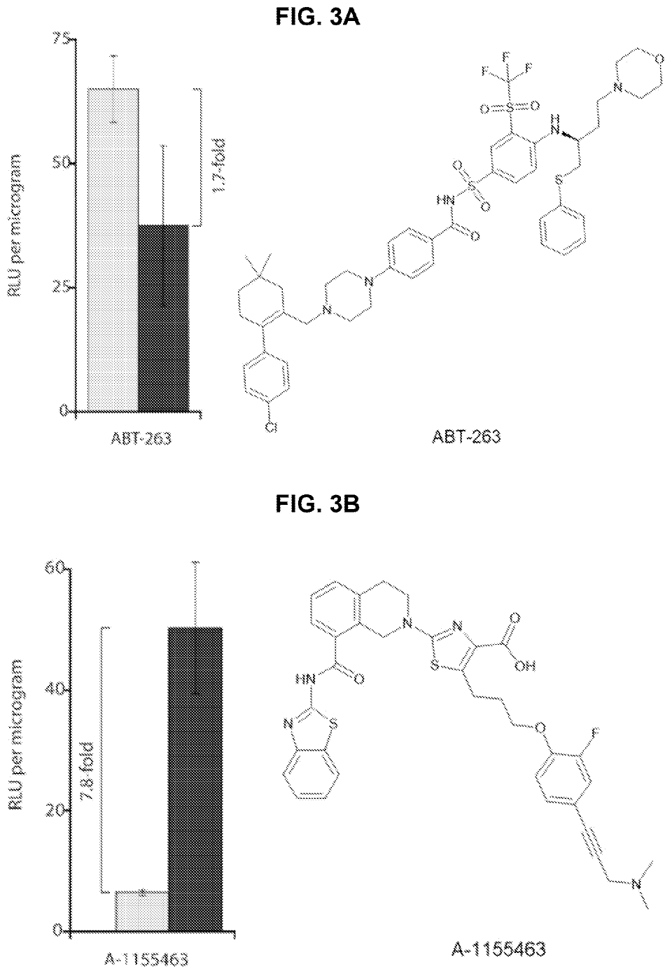

Having successfully demonstrated that it is possible to fragment a protein and reassemble it using a known ligand, multiple small molecule ligands were tested for the induced dimerization of Bcl-xL 209 R139A to see if there was a difference in dose-response or signal. One inhibitor of Bcl-xL, Abbvie's A-1155463, did not induce significant reassembly despite being known to bind with sub-nanomolar affinities in the literature. The R139A construct used may not have been binding as tightly to the small molecule as the parent. To test this, Bcl-xL 209 R139A was assayed side-by-side with its wild-type parent, which was also fragmented to compare the small-molecule ligands ABT-263 and A-1155463. The split-luciferase signal from the two different split proteins showed no significant difference for ABT-263 ligand-induced reassembly but showed significant differences in selectivity for A-1155463 with preferential binding for wild-type C-Bcl-XL 113-209 over the R139A mutant of C-Bcl-XL 113-209 (FIG. 3). This unanticipated selectivity from the R139A mutant may allow for engineering orthogonal CID systems by tuning the small molecule and protein partner.

The newly identified CIDs were tested for reassembling a functional protein tyrosine kinase, Src. As previously mentioned, prior studies have generated split-kinases and shown that they can be reassembled in cells using the rapamycin, abscisic acid, and gibberellic acid systems.

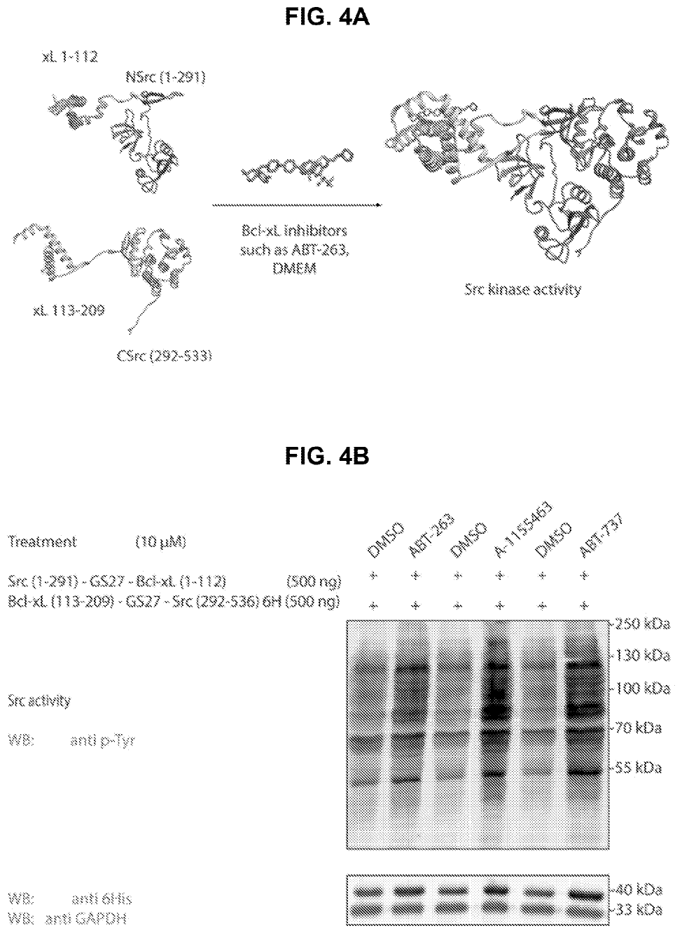

The most ligand-responsive fragments, N-Bcl-xL 1-112 and C-Bcl-XL WT 113-209, were cloned into existing full-length split-Src constructs. N-Bcl-xL was attached via a 25-residue linker to the N-terminus of N-Src. C-Bcl-XL WT was attached via a 25-residue linker to C-Src Y530F (FIG. 4A). Each split-kinase system was tested in HEK293T cells via transient transfection and treated with either DMSO or 10 UM of Bcl-xL ligands A-1155463, ABT-263, or ABT-737. Each Bcl-xL inhibitor tested generated significant increases in global tyrosine phosphorylation (FIG. 4B). Thus, the new ligand-gated split-Bcl-xL system is demonstrated to be portable and can clearly be used to control a variety of split-proteins in cells. More importantly, this novel CID system can likely be used in combination with the three existing CID systems to generate additional layers of control in cells for controlling pathways with externally added small molecules. For example, a split kinase and a corresponding split phosphatase may be used in conjunction with a split target protein (e.g., as described herein) to examine any given phosphorylation pathway in a cell. In some embodiments, the split target protein may be either upstream or downstream of a phosphorylation cascade (e.g., the split kinase). The aforementioned system would allow precise signaling studies to be undertaken by titration of two or more ligands (e.g., small molecules; e.g., one ligand for each split protein in the system).

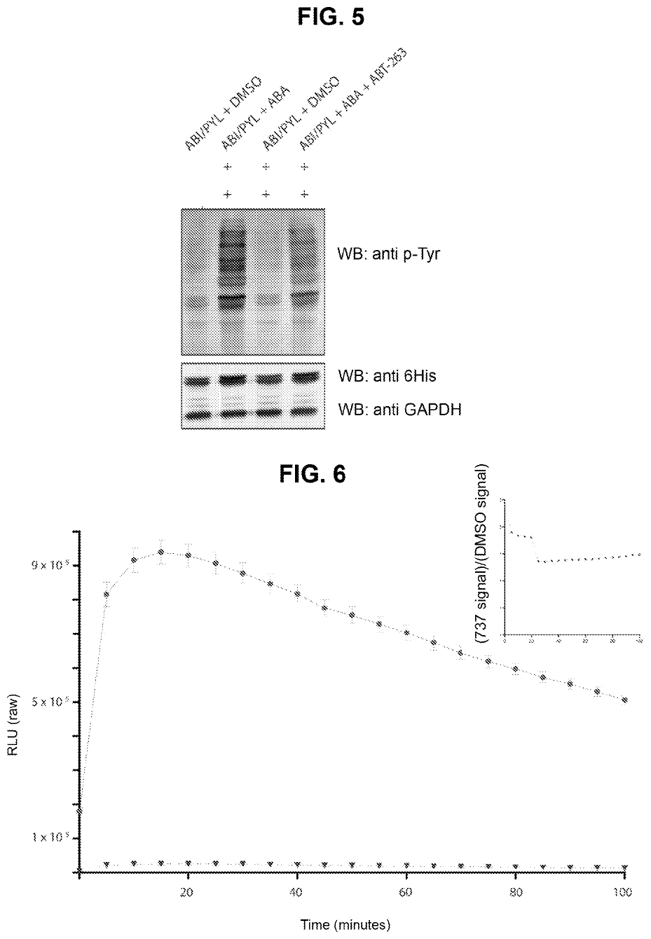

A lower intensity of induced phosphorylation in the ABT-263 treatment was observed compared to A-1155463 and ABT-737. It was hypothesized that there might be off-target kinase inhibition by ABT-263, which is not present in the other ligands tested. To interrogate this potential off-target kinase inhibition, an orthogonal split-Src construct design was employed with ABI and PYL domains and the CID, abscisic acid. The split-Src was tested as fragments without CID, fragments with CID, and fragments with CID plus ABT-263. In the presence of the abscisic acid CID alone, the ABI/PYL Src shows strong phosphorylation; interestingly, upon the addition of ABT-263 to this CID system, phosphorylation is slightly decreased (FIG. 5).

Interestingly, the specific Bcl-xL CID system inadvertently identified a possible mechanism for molecular selectivity through an R139A mutation. Several additional inhibitors to Bcl-xL exist, and there may be significant potential for inhibitor: mutant pairs that are orthogonal to their cellular binders and can be used as selective CIDs inside mammalian cells. There may be one or more permutations that would yield two or more concurrent protein switches within the cell to control different proteins in a spatiotemporal fashion. CID tools enable localization and proximity to be investigated and controlled, and the utility of novel CID scaffolds is potentially significant. It is anticipated that this approach for CID generation is likely generalizable and can be extended to many different protein: ligand complexes and represents a potentially significant advance in protein engineering technology as CIDs are used for many different applications.

For example, a ligand may induce the localization of a target protein by tethering said protein somewhere in the cell upon the addition of said ligand. Without wishing to limit the present invention, it is believed that induced localization by the ligand allows for additional regulation of the target protein (e.g., extracellular or nuclear localization, membrane binding, or trafficking).

### Experimental

Small molecule ligands A-1155463 (APExBIO), ABT-737 (Selleck) and ABT-263 (Selleck) were purchased. Cloning was performed by PCR using Kapa Hi-Fi (KapaBiosystems, Roche). Restriction enzymes, dNTPs, HiT4 Ligase, and Taq ThermoPol polymerase were purchased from NEB. Sequencing was performed at the University of Arizona Genomics Core. HEK293T cells (ATCC) were cultured in complete media consisting of DMEM (Cytiva HyClone), 10% FBS (Corning), 100 U penicillin per mL, and 100 μg streptomycin per mL (Cytiva HyClone), and 2.5 μg amphotericin B per mL (Cytiva HyClone) under 5% CO2 atmosphere. 96-well culture plates and luminescence plates were manufactured by Greiner, and 6-well culture plates were manufactured by Nunc. L-GLO substrate mixture for luminescence assays was manufactured by Luceome Biotechnologies™.

The initial sites were identified via alignment of human Bcl-xL (UniProt ID Q07817) and five human homologs: BAK (Q16611), Bcl-2 (P10415), Bcl-W (Q92843), Bfl-1 (Q16548), and Mcl-1 (Q07820). To further reinforce site identification and to potentially identify more sites, manual searches were performed on UniProt as well as BLAST searches to find each of the cytoplasmic proteins (i.e., Bcl-2, Bcl-xL, Bcl-W, Bfl-1, and Mcl-1) in as many species as possible. All sequences were managed and analyzed using BioEdit. Sequences were aligned in BioEdit using the ClustalW function.

Low passage (≤25) cells were seeded onto T-75 (Fisher) flasks at 1E6 cells per mL and grown for 24 hours in complete media. Transient transfections were performed using PolyJet (SignaGen). 96-well culture plates were coated with 50 μL/well of 50 μg/mL solution of poly-D-lysine (MilliPore) and incubated at 4C overnight. Cells were transfected for ˜16 h, then were trypsinized and quantified. Cells were plated onto coated 96-well plates at 1E4 cells/well in DMEM supplemented with 0.5% v/v FBS. Treatment was dissolved in DMSO and added to a final DMSO concentration of 0.05% by volume. Cells were grown in treatment media for 24 h before being lysed with an in-house detergent-based buffer. Cell lysis was clarified via centrifugation, and 50 μL of clarified lysate was plated per well on a white U-bottom luminescence plate. 80 μL of L-GLO was added to each well, and the plate was incubated at room temperature for 25-40 minutes before luminescence was read on a Centro XS3 LB960 (Berthold Technologies) microplate luminometer. Separately, clarified lysate was quantified using a Micro-BCA kit (Pierce), and luminescence was normalized to RLU per microgram of total protein using Microsoft Excel. All data presented were plotted using Kaleidagraph (Synergy) 3.5.

A consistent pattern with this split protein system was observed where both signal and signal: background ratios markedly changed over time. To test this, the split protein system was incubated with L-GLO and took readings of both signals (treatment with ABT-737) and background (treatment with DMSO only) at five-minute intervals over a 100-minute time frame. This change in raw signal and signal: background ratios was independent of L-GLO degradation (data not shown).

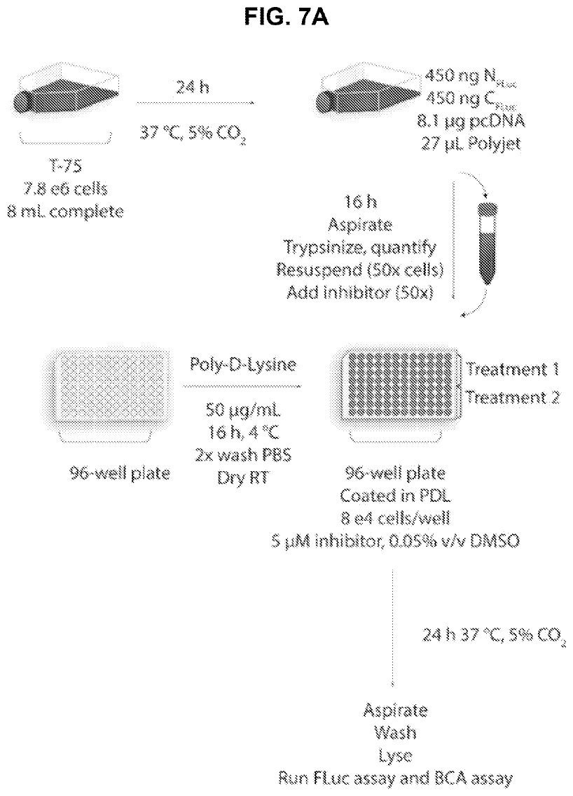

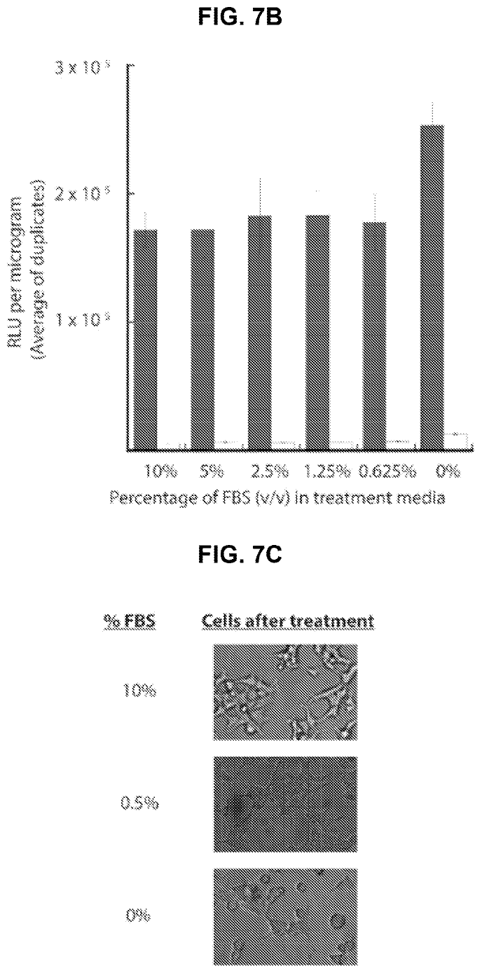

Initial iterations of this assay were performed by replating cells and treating in complete media (10% FBS). It was questioned if this was the most generalizable method, given that many small molecules exhibit the potential to bind serum proteins and that serum binding of the potential CIDs may impact the selectivity results. To test this, a step down of FBS was performed at the treatment media step of the assay (FIG. 7A). It began with the expectation that FBS percentage over the 24-hour treatment incubation would impact the total signal, and that a lower FBS percentage would give a lower FLuc signal due to effects on the translation rates of the split-protein constructs. Contrary to predictions, no significant changes were observed in signal between any of the conditions that contain FBS (FIG. 7B). Separately, cells treated in several treatment concentrations were photographed before lysis and discovered that the no-FBS condition changed HEK293T cell morphology (FIG. 7C). Based upon these findings, and with the minimal difference between the signal at 0% FBS and <1% FBS, a 0.5% v/v FBS treatment media was used for the remaining assays. All data presented in the results used 0.5% v/v FBS treatment media.

Based upon the success of the ligand-dependent design of a split-Bcl-xL system, it was hypothesized that many known protein: ligand complexes may be re-engineered into ligand gated split-protein systems. The ligand gated split-Bcl-XL serves as the first proof of concept; however, utilizing systems in mammalian cells using non-mammalian proteins and their ligands may be optimal. Without wishing to limit the present invention to any theory of mechanism, it is believed that the use of non-mammalian proteins and their ligands to create a split protein system, as described herein, would allow for mammalian cell signaling pathways to be studied without perturbing said pathways upon activation of the non-mammalian split protein system.

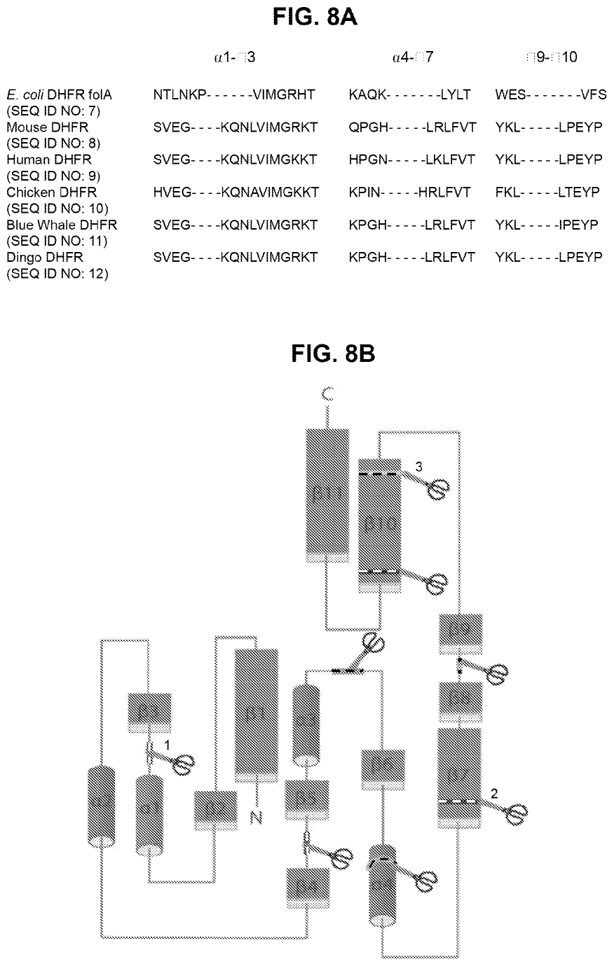

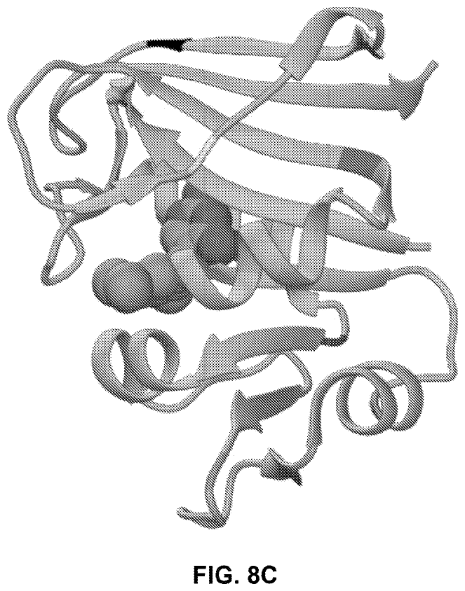

Thus, a ligand-gated E. coli dihydrofolate reductase (eDHFR) will be created next, which binds selectively to ligands such as trimethoprim or iclaprim with low nanomolar affinities. 200 DHFR sequences from unique species were aligned using UniProt and the BioEdit software. This identified several non-homologous regions in the ClustalW sequence alignment (FIG. 8A). These non-homologous regions were then mapped to both topology diagrams (FIG. 8B) and the three-dimensional structure of eDHFR in complex with trimethoprim (FIG. 8C). It was predicted that one or more of the eight proposed split-eDHFR systems would show ligand dependent reassembly and can be used to gate the activity of other proteins as has been demonstrated for existing CID systems. Based upon the successes in using split-Bcl-xL to control enzyme complementation in human cells, this strategy may be potentially generalizable to many known protein: ligand pairs in literature.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A split protein system, wherein a first protein is split into a first protein fragment and a second protein fragment, wherein a target protein is split into a first target fragment and a second target fragment, wherein the split protein system comprises:
a) a first split protein complex comprising the first target fragment operatively linked to the first protein fragment;
b) a second split protein complex comprising the second target fragment operatively linked to the second protein fragment; and
c) a ligand capable of chemically inducing dimerization of the first split protein complex and the second split protein complex by operatively linking the first protein fragment and the second protein fragment, wherein the ligand interacts with the first protein fragment and the second protein fragment to modulate the activity of the target protein.

2. The system of claim 1, wherein the first protein comprises a plurality of split sites, wherein the first protein is split at at least one of the split sites.

3. The system of claim 1, wherein the ligand is a small molecule ligand.

4. The system of claim 1, wherein the ligand is an inhibitor of the first protein.

5. The system of claim 1, wherein the first target fragment is operatively linked to the first protein fragment via a linker.

6. The system of claim 1, wherein the second target fragment is operatively linked to the second protein fragment via a linker.

7. The system of claim 1, wherein the first protein fragment comprises an N-terminus of the first protein, and the first target fragment comprises an N-terminus of the target protein, wherein the first protein fragment is linked to the N-terminus of the first target fragment.

8. The system of claim 1, wherein the second protein fragment comprises a C-terminus of the first protein and the second target fragment comprises a C-terminus of the target protein, wherein the second protein fragment is linked to the C-terminus of the second target fragment.

9. The system of claim 1, wherein the first protein comprises a Bcl-xL protein.

10. The system of claim 9, wherein the first protein fragment is N-Bcl-xL 1-112 and the second protein fragment is C-Bcl-XL WT 113-209.

11. The system of claim 9, wherein the ligand is a Bcl-xL ligand.

12. The system of claim 11, wherein the Bcl-xL ligand is A-1155463, ABT-263, or ABT-737.

13. The system of claim 9, wherein the Bel-xL protein is mutant Bcl-xL R139A.

14. The system of claim 1, wherein the target protein is any functional protein.

15. The system of claim 1, wherein the target protein is a tyrosine kinase.

16. (canceled)

17. A method of chemically induced dimerization of a target protein, comprising:
a) operatively linking a first protein fragment from a first protein to a first target fragment from a target protein to form a first split protein complex;
b) operatively linking a second protein fragment from the first protein to a second target fragment from the target protein to form a second split protein complex; and
c) contacting the first split protein complex and the second split protein complex with a ligand, wherein the ligand chemically induces dimerization of the first split protein complex and the second split protein complex by operatively linking the first protein fragment and the second protein fragment.

18. (canceled)

19. A method for multimerizing a target protein in cells, the method comprising:
a) providing cells which contain:
i. a first recombinant nucleic acid encoding a first chimeric protein, wherein the first chimeric protein comprises a first protein fragment operatively linked to a first target fragment; and
ii. a second recombinant nucleic acid encoding a second chimeric protein, wherein the second chimeric protein comprises a second protein fragment operatively linked to a second target fragment; and
b) contacting the cells with a ligand; wherein the ligand chemically induces multimerization of the first chimeric protein and the second chimeric protein by operatively linking the first protein fragment and the second protein fragment.

20. The method of claim 19, wherein the cells are mammalian cells.

21. The method of claim 19, wherein the cells are present in a whole organism and contacting the cells with the ligand comprises administering the ligand to the organism.

22. The method of claim 21, wherein the organism is a mammal.


SEQUENCE LISTING (28 sequences)

SEQ ID NO: 1 - protein, 28 aa, Homo sapiens
LHITPGTARDGVNWGRIVQVLVSRIAAW

SEQ ID NO: 2 - protein, 28 aa, Homo sapiens
LQPTAENAESGINWGRVVTGFLGQVTRF

SEQ ID NO: 3 - protein, 33 aa, Homo sapiens
VNVVSVDTAEDGIINWGRIVAPDVDTYKEISYF

SEQ ID NO: 4 - protein, 28 aa, Homo sapiens
LHLTPFTARDGVNWGRIVSPLVDNIALW

SEQ ID NO: 5 - protein, 28 aa, Homo sapiens
LHVTPGSAQGGPNWGRLVEPLVGQVQEW

SEQ ID NO: 6 - protein, 29 aa, Homo sapiens
LDIKNEDDSDGVTNWGRIVESCIEPLAES

SEQ ID NO: 7 - protein, 27 aa, Homo sapiens
NTLNKPVIMGRHTKAQKLYLTWESVFS

SEQ ID NO: 8 - protein, 33 aa, Homo sapiens
SVEGKQNLVIMGRKTQPGHLRLFVTYKLLPEYP

SEQ ID NO: 9 - protein, 33 aa, Homo sapiens
SVEGKQNLVIMGKKTHPGNLKLFVTYKLLPEYP

SEQ ID NO: 10 - protein, 33 aa, Homo sapiens
HVEGKQNAVIMGKKTKPINHRLFVTFKLLTEYP

SEQ ID NO: 11 - protein, 33 aa, Homo sapiens
SVEGKQNLVIMGRKTKPGHLRLFVTYKLIPEYP

SEQ ID NO: 12 - protein, 33 aa, Homo sapiens
SVEGKQNLVIMGRKTKPGHLRLFVTYKLLPEYP

SEQ ID NO: 13 - DNA, 1665 bp, synthetic construct
  source 1..1665: mol_type = other DNA
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatgcggccgcggcggttcaggcggtgggggttctggcgggggtgggagcgtcgacggcggttcaggcggtcaattggatatcatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctc

SEQ ID NO: 14 - DNA, 879 bp, synthetic construct
  source 1..879: mol_type = other DNA
atgcacatcaccccagggacagcatatcagagctttgaacaggtagtgaatgaactcttccgggatggggtaaactggggtgcgattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcgaattcgagctcggcgcgcctgcaggtcgacaagcttgcggccgcggcggttcaggcggtgggggttctggcgggggtgggagcgtcgacggcggttcaggcggtcaattggatatcggccggccaatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattg

SEQ ID NO: 15 - protein, 555 aa, synthetic construct
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGCGRGGSGGGGSGGGGSVDGGSGGQLDIMSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQL

SEQ ID NO: 16 - protein, 293 aa, synthetic construct
MHITPGTAYQSFEQVVNELFRDGVNWGAIVAFFSFGGALCVESVDKEMQVLVSRIAAWMATYLNDHLEPWIQENGGWDTFVELYGNNAAAESRKGQEREFELGAPAGRQACGRGGSGGGGSGGGGSVDGGSGGQLDIGRPMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 17 - DNA, 1734 bp, synthetic construct
  source 1..1734: mol_type = other DNA
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatgcggccgcggcggttcaggcggtgggggttctggcgggggtgggagcgtcgacggcggttcaggcggtcaattggatatcatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggta

SEQ ID NO: 18 - DNA, 810 bp, synthetic construct
  source 1..810: mol_type = other DNA
atgaactggggtgcgattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcgaattcgagctcggcgcgcctgcaggtcgacaagcttgcggccgcggcggttcaggcggtgggggttctggcgggggtgggagcgtcgacggcggttcaggcggtcaattggatatcggccggccaatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattg

SEQ ID NO: 19 - protein, 578 aa, synthetic construct
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGCGRGGSGGGGSGGGGSVDGGSGGQLDIMSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGV

SEQ ID NO: 20 - protein, 270 aa, synthetic construct
MNWGAIVAFFSFGGALCVESVDKEMQVLVSRIAAWMATYLNDHLEPWIQENGGWDTFVELYGNNAAAESRKGQEREFELGAPAGRQACGRGGSGGGGSGGGGSVDGGSGGQLDIGRPMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 21 - DNA, 1821 bp, synthetic construct
  source 1..1821: mol_type = other DNA
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatgcggccgcggcggttcaggcggtgggggttctggcgggggtgggagcgtcgacggcggttcaggcggtcaattggatatcatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtgcgattgtggcctttttctccttcggcggggcactgtgcgtggaaagcgtagacaaggagatgcaggtattggtgagt

SEQ ID NO: 22 - DNA, 723 bp, synthetic construct
  source 1..723: mol_type = other DNA
atgcggatcgcagcttggatggccacttacctgaatgaccacctagagccttggatccaggagaacggcggctgggatacttttgtggaactctatgggaacaatgcagcagccgagagccgaaagggccaggaacgcgaattcgagctcggcgcgcctgcaggtcgacaagcttgcggccgcggcggttcaggcggtgggggttctggcgggggtgggagcgtcgacggcggttcaggcggtcaattggatatcggccggccaatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattg

SEQ ID NO: 23 - protein, 607 aa, synthetic construct
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGCGRGGSGGGGSGGGGSVDGGSGGQLDIMSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGAIVAFFSFGGALCVESVDKEMQVLVS

SEQ ID NO: 24 - protein, 241 aa, synthetic construct
MRIAAWMATYLNDHLEPWIQENGGWDTFVELYGNNAAAESRKGQEREFELGAPAGRQACGRGGSGGGGSGGGGSVDGGSGGQLDIGRPMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 25 - DNA, 1296 bp, synthetic construct
  source 1..1296: mol_type = other DNA
atgggtagcaacaagagcaagcccaaggatgccagccagcggcgccgcagcctggagcccgccgagaacgtgcacggcgctggcgggggcgctttccccgcctcgcagacccccagcaagccagcctcggccgacggccaccgcggccccagcgcggccttcgcccccgcggccgccgagcccaagctgttcggaggcttcaactcctcggacaccgtcacctccccgcagagggcgggcccgctggccggtggagtgaccacctttgtggccctctatgactatgagtctaggacggagacagacctgtccttcaagaaaggcgagcggctccagattgtcaacaacacagagggagactggtggctggcccactcgctcagcacaggacagacaggctacatccccagcaactacgtggcgccctccgactccatccaggctgaggagtggtattttggcaagatcaccagacgggagtcagagcggttactgctcaatgcagagaacccgagagggaccttcctcgtgcgagaaagtgagaccacgaaaggtgcctactgcctctcagtgtctgacttcgacaacgccaagggcctcaacgtgaagcactacaagatccgcaagctggacagcggcggcttctacatcacctcccgcacccagttcaacagcctgcagcagctggtggcctactactccaaacacgccgatggcctgtgccaccgcctcaccaccgtgtgccccacgtccaagccgcagactcagggcctggccaaggatgcctgggagatccctcgggagtcgctgcggctggaggtcaagctgggccagggctgctttggcgaggtgtggatggggacctggaacggtcaagcttgcggccgcggcggttcaggcggtgggggttctggcgggggtgggagcgtcgacggcggttcaggcggtcaattggatatcatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctc

SEQ ID NO: 26 - DNA, 1128 bp, synthetic construct
  source 1..1128: mol_type = other DNA
atgcacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcggcggccgcggcggttcaggcggtgggggttctggcgggggtgggagcgtcgacggcggttcaggcggtcaattggatatcaccaccagggtggccatcaaaaccctgaagcctggcacgatgtctccagaggccttcctgcaggaggcccaggtcatgaagaagctgaggcatgagaagctggtgcagttgtatgctgtggtttcagaggagcccatttacatcgtcacggagtacatgagcaaggggagtttgctggactttctcaagggggagacaggcaagtacctgcggctgcctcagctggtggacatggctgctcagatcgcctcaggcatggcgtacgtggagcggatgaactacgtccaccgggaccttcgtgcagccaacatcctggtgggagagaacctggtgtgcaaagtggccgactttgggctggctcggctcattgaagacaatgagtacacggcgcggcaaggtgccaaattccccatcaagtggacggctccagaagctgccctctatggccgcttcaccatcaagtcggacgtgtggtccttcgggatcctgctgactgagctcaccacaaagggacgggtgccctaccctgggatggtgaaccgcgaggtgctggaccaggtggagcggggctaccggatgccctgcccgccggagtgtcccgagtccctgcacgacctcatgtgccagtgctggcggaaggagcctgaggagcggcccaccttcgagtacctgcaggccttcctggaggactacttcacgtccaccgagccccagttccagcccggggagaacctccaccatcaccatcaccat

SEQ ID NO: 27 - protein, 432 aa, synthetic construct
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGQACGRGGSGGGGSGGGGSVDGGSGGQLDIMSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQL

SEQ ID NO: 28 - protein, 376 aa, synthetic construct
MHITPGTAYQSFEQVVNELFRDGVNWGRIVAFFSFGGALCVESVDKEMQVLVSRIAAWMATYLNDHLEPWIQENGGWDTFVELYGNNAAAESRKGQERGGRGGSGGGGSGGGGSVDGGSGGQLDITTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQFQPGENLHHHHHH